(19) United States
(12) Patent Application Publication
(10) Pub. No.: US 2010/0317079 A1
(43) Pub. Date: Dec. 16, 2010

(54) SILAGE WASTE RECOVERY PROCESS, FACILITY, SYSTEM, AND PRODUCT

(76) Inventors: Bertrand Lesperance, Sallaberry de Valleyfield (CA); James Anglehart, Montreal (CA)

Correspondence Address: HAHN LOESER & PARKS, LLP, One GOJO Plaza, Suite 300, AKRON, OH, 44311-1076, US

(21) Appl. No.: 12/521,434

(22) PCT Filed: Jun. 11, 2008
(86) PCT No.: PCT/CA08/01095

§ 371 Date: Jun. 26, 2009

Related U.S. Application Data
(60) Provisional application No. 61/044,587, filed on Apr. 14, 2008.
(60) Provisional application No. 60/929,115, filed on Jun. 13, 2007.

(51) Int. Cl. C12P 7/08; C12M 1/12; C12P 7/02

(52) U.S. Cl. 435/163; 435/297.1; 435/155

(57) ABSTRACT

A silo of conventional construction is provided with a collection pan at its bottom to collect effluent. The effluent can be stored and/or processed. Processing can include separation of water to concentrate the effluent. The effluent can contain fermentation product, such as alcohol, generated within the silo prior to collection, and it can also be fermented after collection. Processed effluent can be handled safely without risk of contamination to ground water at the silo site.

### TECHNICAL FIELD

The present specification relates to the field of handling agricultural waste, and more specifically to a process and a facility for utilizing the waste liquid from silos, silage, haylage and balage.

### BACKGROUND

Silos are used on farms to store silage such as corn, hay, or soya for feeding livestock. A silo is not merely a storage facility for crops, but is rather a structure that facilitates the silage fermentation process that makes crops more digestible for farm animals. Basic information on the silage process is found at http://en.wikipedia.org/wiki/Silage. The harvested crop contains a high level of water, and the water content can vary from crop to crop, the time of harvest, as well as due to weather/irrigation conditions at the time that the crop is harvested. When stored in a silo, gravity causes high pressure on the silo contents at the bottom of the full silo and a combination of this pressure and the effect of gravity over time causes a high percentage of the water to drip out of the silo contents or silage. The silage thus generates liquid effluent waste (“silage juice”) during storage and this liquid collects at the bottom of the silo. The rate of liquid generation is surprisingly fast in the days and weeks following harvest and initial storage in the silo. The volume of seepage is varies with the moisture content of the crop, and can be from about 300 L to 700 L per ton. Normally a drain is provided at the bottom of the silo for keeping the bottom dry, and an overflow drain pipe may lead from the drain area to an auxiliary drain area next to the silo. The liquid waste is generally toxic however and pollutes the ground and water with which it comes in contact. In many cases, the silo is not watertight, and a portion of this liquid does not go into the central drain pit. Instead, the liquid level rises and leaks from the silo side wall into the ground at the silo foundation.

The silage juice contains a variety of compounds in sufficient concentration to be a problem (e.g. toxic) if the juice is released directly into the ground at the silo location.

A variety of solutions have been proposed to reduce the amount of or treat silage seepage or juice. For example, it has been proposed to add the seepage to manure that will be distributed as fertilizer. This approach attempts to reduce the problem of contamination by spreading out the surface area subjected to the silage juice.

It is well known to produce ethanol from corn. Many farming communities are now in the process of obtaining an ethanol producing facility and are growing corn to supply the facility with the raw product to make ethanol. Ethanol is produced from fermentation of a corn mash. Ethanol can be added to gasoline in an amount of up to 10% without requiring a change in the vehicle's engine (for engines manufactured after 1980). The addition of ethanol increases the fuel octane rating and is presently being encouraged by government authorities to reduce the dependence on petroleum for transportation fuel. However, the use of corn and grain for fuel purposes is presently driving the price of corn higher, and thus causing an increase in the price of grains and meat raised using grain products.

### SUMMARY

A silo of conventional construction is provided with a collection pan at its bottom to collect effluent. The effluent can be stored and/or processed. Processing can include separation of water to concentrate the effluent. The effluent can contain fermentation product, such as alcohol, generated within the silo prior to collection, and it can also be fermented after collection. Processed effluent can be handled safely without risk of contamination to ground water at the silo site.

It is known that the silage seepage arising from the ensilage process in farm silos is rich in nutrients, but also exhibits a very high biological oxygen demand (BOD). It is also known that this seepage waste can be quite toxic to the environment, particularly groundwater and surface-water environments. Applicant has conducted analytical tests on silage effluent and determined that raw silage effluent contains a variety of alcohols the sum of which is approximately 3 g/L in the case of whole corn silage having approximately 35% moisture at harvest (harvested in late August in southern Québec, Canada). The level of alcohol was measured from samples taken within a few weeks of harvest. The alcohols found were mostly ethanol (more than half), with 1-propanol and 1-butanol comprising most of the rest of the constituents. BOD from the samples were approximately 60,000 mg/L.

While some embodiments are described herein with respect to silage effluent, it will be understood that by silage effluent, silage seepage, silage liquid, and silage juice, it is meant the effluent liquid from silos, stacks, and bales of harvested plants and crops (such as hay, corn, alfalfa, wheat, etc.) whether such items are subjected to conditions of fermentation for the purposes of causing a transformation of the vegetable matter into high moisture content animal fodder or merely for crop storage purposes. It will likewise be understood that by the term “silo” is it intended to mean any stack, bale, loft, tower silo or other suitable structure.

In accordance with an embodiment, the silage seepage from a silo is collected from a silo drain and directed into a suitable holding tank, rather than being dumped into the local environment. It has been recognized that the high nutrient content of this silage seepage makes this waste product suitable for microbially mediated treatment (i.e. bioconversion) and/or downstream processing into a value product. As such, according to some embodiments, the silage seepage is used as a substrate/nutrient source for fermentation technology, such as for the production of ethanol.

In accordance with other embodiments, the silo seepage from a silo is passed through a reverse osmosis (RO) filter to separate a portion of the water from the toxic products in the water. This filtering can be performed at the silo site. The water can be stored for irrigation or returned to the soil without the adverse effects known with conventional draining of silo juice. The cost of the RO process is recovered many times over by collecting the concentrated silo juice and processing the juice by fermentation to produce ethanol or other useful products.

The process substantially reduces pollution around the farm and provides the additional benefit of producing a desirable product, such as ethanol fuel. The silage waste becomes a useful by-product from the ensilage process. This additional benefit is gained from what is normally a waste product. For example, the use of silage seepage to create alcohol, and in particular ethanol may also serve to alleviate growing objections to the practice of growing corn specifically for the production of ethanol, which opponents argue is a wasteful use of good farm land. Thus, the supply of ethanol fuel from farms is maintained (or even increased) without needing to divert as much crops from animal feed to fuel production. Furthermore, dairy and meat production farms that grow their own fodder can begin to produce an ethanol fuel source without needing to grow crops suitable for ethanol production or to divert fodder crops to the production of ethanol fuel. Thus the cost of food production may remain less tied to the value of crops for fuel production.

According to some embodiments, there is provided is a process for utilizing waste from silage comprising the steps of storing silage in a silo; collecting the liquid waste generated from the silage from the bottom of the silo; and processing the liquid waste by fermentation to produce a value product. Optionally, concentration of the liquid waste can be done by reverse osmosis (RO) or any other suitable filtration or separation method so that decontaminated water is made available and a richer liquid waste product can be processed.

According to a further aspect of an embodiment, there is provided is a facility for obtaining fermentation product from silage comprising a silage silo having a structure to capture effluent with an outlet; a storage tank; and a drain line from the outlet to the storage tank. More than one silo can be connected to the storage tank.

According to a further aspect of an embodiment, provided is a facility for obtaining a fermentation product from silage comprising a silage silo having a drain, a storage tank adjacent the silo, and a drain line from the drain to the storage tank, wherein silage seepage produced in the silage silo is collected in the storage tank and subjected to conditions conducive to fermentation.

According to an embodiment, provided is a facility for obtaining a fermentation product from silage comprising at least one silage silo having a bottom, a collector at the bottom for collecting a silage juice, and one of a storage tank and separation device in communication with an outlet of the collector.

According to a further aspect of an embodiment, provided is a facility further comprising an overflow tank for collecting overflow seepage from the storage tank.

According to a further aspect of an embodiment, provided is a facility wherein the storage tank is located underground.

According to a further aspect of an embodiment, provided is a facility wherein the facility comprises a reverse osmosis separation device to separate usable water from the silage juice.

According to a further aspect of an embodiment, provided is a facility wherein the drain line comprises a filter assembly for filtering the seepage prior to collection in the storage tank.

According to a further aspect of an embodiment, provided is a facility wherein the storage tank is provided with environmental controls for promoting proper fermentation conditions in the storage tank.

According to a further aspect of an embodiment, provided is a facility where the environmental controls comprise at least one of temperature control, stirring and gas perfusion.

According to a further aspect of an embodiment, provided is a facility wherein the collector comprises a floor pan covering a bottom aperture of a tower silo to prevent egress of seepage from the silo into the surrounding foundation soil.

According to a further aspect of an embodiment, provided is a facility wherein at least one additional pump is provided to move the seepage to the storage tank.

According to a further aspect of an embodiment, provided is a facility further comprising a central processing unit for providing automated control to at least portions of the facility.

According to a further aspect of an embodiment, provided is a facility further comprising a secondary drain at a higher position on the silo for indicating seepage backup and accumulation within the silo.

According to an embodiment, provided is a process for treating silage seepage comprising the steps of collecting the silage seepage generated in a silo, and processing the silage seepage by one of fermentation and separation to produce a value product.

According to a further aspect of an embodiment, provided is a process wherein the value product is alcohol.

According to a further aspect of an embodiment, provided is a process wherein the value produce is ethanol.

According to a further aspect of an embodiment, provided is a process wherein the process comprises the addition of starter cultures.

According to a further aspect of an embodiment, provided is a process wherein the process comprises the addition of hydrolyzing enzymes.

According to a further aspect of an embodiment, provided is a process wherein the process comprises the addition of at least one of sugars, carbohydrates and nutrients to promote the process.

According to a further aspect of an embodiment, provided is a process further comprising the step of diluting the seepage prior to fermentation.

According to a further aspect of an embodiment, provided is a process wherein the step of diluting is accomplished using clean water.

According to a further aspect of an embodiment, provided is a process wherein the step of diluting is accomplished using liquid waste derived from downstream processing of the fermented seepage.

According to a further aspect of an embodiment, provided is a process wherein the processing comprises separating the seepage by reverse osmosis filtration to produce water and concentrated seepage.

According to a further aspect of an embodiment, provided is a process wherein the silage seepage collected from the silo contains about 0.1 to 1% alcohol by weight, and alcohol is separated from the concentrated seepage.

According to a further aspect of an embodiment, provided is a process wherein the water is potable.

### BRIEF DESCRIPTION OF THE FIGURES IN THE DRAWINGS

Embodiments will now be discussed, by way of example only, with reference to the attached Figures, in which:

FIG. 1 is a schematic representation of a silo seepage collection and treatment system according to one embodiment;

FIG. 2 is a cross-sectional view of a lower section of a silo detailing the ring-shaped foundation;

FIG. 3A is an alternate embodiment of the system shown in FIG. 1 in which a reverse osmosis separation system is located in the drain line;

FIG. 3B is an alternate embodiment of the system shown in FIG. 1 in which a pump is located in the drain line;

FIG. 4 shows an alternate embodiment of a silo in which a plurality of drains is provided;

FIG. 5 shows a further alternate embodiment in which a secondary overflow tank is provided; and

FIG. 6 shows a further alternate embodiment in which collection of the seepage waste is governed by a centrally controlled valve.

### DETAILED DESCRIPTION

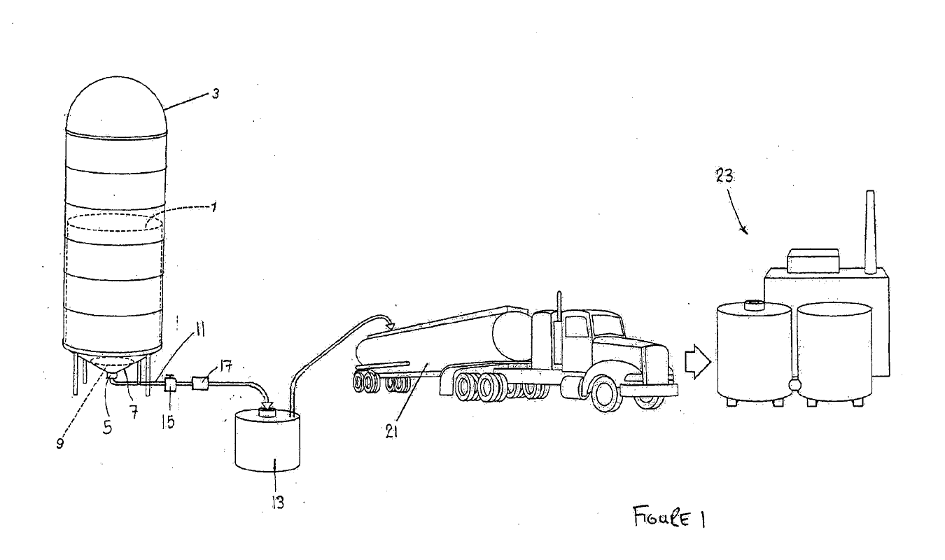

Turning now to FIG. 1, shown is a general arrangement of a particular embodiment. As represented, the corn silage tissue or other forage material 1 is stored in a silo (e.g. tower silo) 3 on a farm to promote ensilage, thereby providing feed for the livestock. To control and collect silage seepage 9 arising from the ensilage process, the silo 3 is configured with at least one drain 5 located in the vicinity of the bottom 7 of the silo 3. In the particular embodiment shown, the drain 5 is located towards the bottom of a conical lower section of the silo 3. The drain 5 is connected to a drain line 11 that leads to a storage tank 13. To facilitate drainage, the storage tank 13 is preferably located at a lower elevation than the silo 3, and in particular the drain 5. For example, the storage tank 13 could be located underground. To control flow of silage seepage, the drain line is preferably provided with a suitable valve 15. At least one removable filter 17 is preferably provided in the drain line 11 to filter out solid debris from the waste liquid.

The storage tank 13 can be equipped with a liquid level sensor. This sensor can be used to report locally (e.g. a display or luminous or audio signal) to the user what the liquid level is, or whether the tank is ready to be emptied, e.g. 80% full. Alternatively or additionally, the report can be done over a telecommunications connection to reach a monitoring center associated with a network of silo installations, so that, for example, a tanker truck can be dispatched to collect the tank liquid.

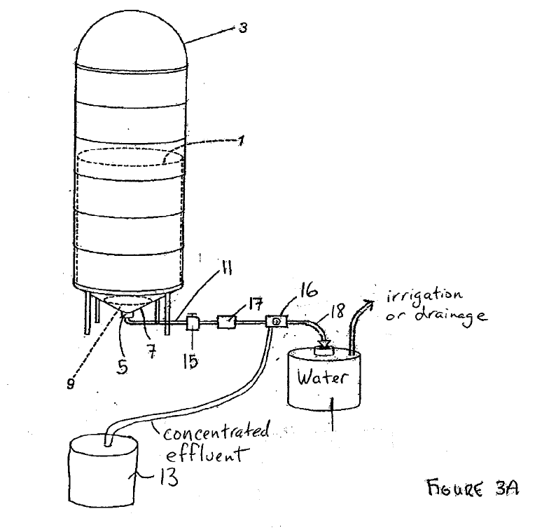

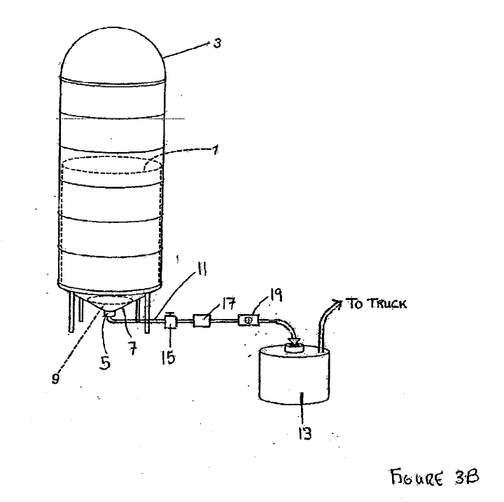

In an alternate configuration shown in FIG. 3A, a reverse osmosis (RO) filter unit 16 is provided downstream of filter 17 (the RO unit can operate in absence of filter 17 as well). Unit 16 includes a pump to raise the fluid pressure before passing through the RO membrane. At the downstream side of the membrane, water is expelled to an outlet 18, while the silage juice remains on the upstream side. The concentrated silage juice from the upstream side is then fed to the continuation of drain line 11 to reservoir 13. The tank 13 can be located above ground and use the pressure from unit 16 to deliver the concentrated liquid from one or more silos into the tank.

The water at outlet 18 can be used for irrigation or other use, or it can be returned to the ground to go back to the water table. A separate water reservoir can be provided for storage, if desired. The cost of installation of an RO unit 16 can be offset by the sale of concentrated silage juice to a processing plant that produces a valuable product such as ethanol, or by processing the concentrated silage juice on site to produce a valuable product. As mentioned hereinbelow, the further fermentation process may not be possible for certain silage juices if the concentration is too high. However, in certain embodiments in which the silage juice is concentrated at site, this can be done to advantage to provide a source of usable water at the site, while reducing the required volume for storage of the concentrated silage juice, and any cost for transportation thereof. The concentrated silage juice can then be diluted as required for fermentation or other processing at the processing plant. It will also be appreciated that a ten times concentration of the silage juice would yield a solution containing about 3% of alcohols for whole corn silage. This alcohol can be then separated by distilling or a similar process to yield alcohol of acceptable purity.

Many silos are configured with a ring-shaped concrete foundation. When silos are filled, much of the load is carried by the soil within the center annulus of the ring foundation. For silos configured with a ring-shaped foundation, most of the silage seepage escapes through the center annulus, into the underlying foundation soil. The movement of seepage into the foundation soil has the potential to soften and destabilize the region, drawing into question the overall safety, reliability and stability of the structure. For example, excessive silage seepage into the foundation soil can increase the pore water pressures in saturated clay soils, having the effect of reducing soil shear strength. Chemical reactions may also occur with (or within) the soil, having the further effect of reducing soil strength. In extreme cases, stabilization of the foundation soil can result in catastrophic failure and collapse of the silo.

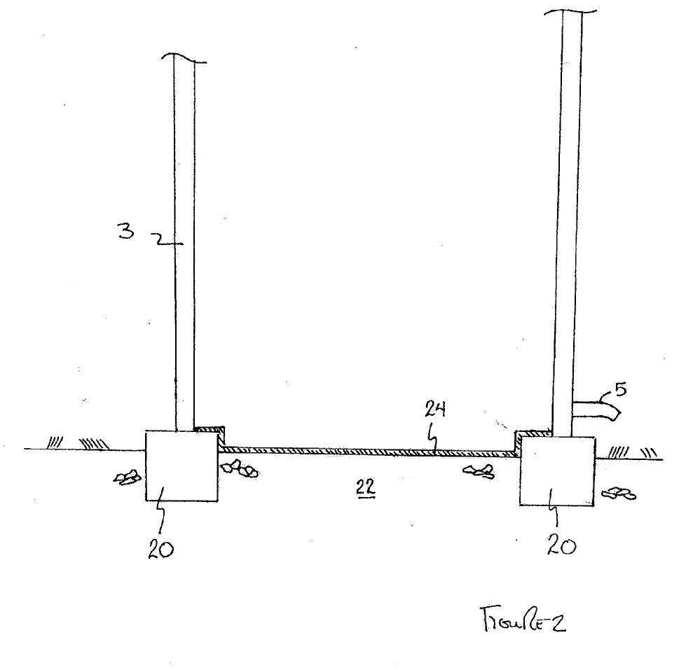

To address the above noted scenario of seepage movement, silo or silos 3 are configured with a discontinuous or ring-shaped foundation 20 should be fitted with a suitable floor pan or membrane 24 to prevent the downward escape of seepage into the foundation soil 22. An example of a silo pan installed in a conventional tower silo is shown in FIG. 2. In this configuration, the drain 5 is located on the side of the silo 3, in the vicinity of the bottom near the ring-shaped foundation. It is presently preferred that the drain be located just above the floor pan, but it will be appreciated that deviations from this preferred configuration are certainly possible.

The silage seepage that is collected in the storage tank 13 presents certain environmental challenges. In particular, due to the very high nutrient content of the seepage, the seepage is potentially quite toxic to groundwater and surface-water environments. As such, government agencies routinely monitor and assert guidelines for dealing with this ensilage waste product. Of particular note is the extremely high BOD of silo seepage, which is very often in excess of 55,000 mg/L. A sample profile of the constituents of silage seepage is presented in Table I

TABLE 1
Constituents of Silage Seepage

| Constituent | Silage Seepage | |
|---|---|---|
| Dry Matter | 5% (2% to 10%) | |
| Total Nitrogen | 1,500 to 4,400 | mg/L |
| Phosphorus | 300 to 600 | mg/L |
| Potassium | 3,400 to 5,200 | mg/L |
| pH | 4.0 (3.6 to 5.5) | |
| Biochemical Oxygen | 12,000 to 90,000 | mg/L |
| Demand (BOD) | | |

Source: Cornell University 1994 and OMAF

In one embodiment, the silage seepage is used as a substrate/nutrient source for fermentation technology. As known in the art, micro-organisms are capable of producing and biotransforming a wide range of products. Fermentation processes are designed to take advantage of these capabilities, and are often used to accomplish the following:
a) production of cells (biomass) such as yeasts;
b) extraction of metabolic products such amino acids, proteins (including enzymes), vitamins, alcohol, etc., for human and/or animal consumption or industrial use such as fertiliser production;
c) modification of compounds (through the mediation of elicitors or through biotransformation); and
d) production of recombinant products.

Alcohol fermentation processes are known, and generally use carbohydrates (sugars, starches, etc.) as feedstock. By using the nutrient rich seepage as fermentation feedstock, a valuable product, for example, ethanol, is derived from what has long been considered a difficult waste product of the ensilage process.

The fermentation stage of the process is able to take on a variety of different configurations. For example, the seepage waste collected in storage tank 13 can be fermented or partially fermented on site, after which it is transported away for subsequent down-stream processing. On site processing can be facilitated by providing environmental controls to the storage tank. For example, to promote fermentation during the fall and winter months, the storage tank can be configured with suitable heaters (e.g. heating pipes and exchangers containing heated water or polyethylene glycol). Alternatively, the contents of the storage tank 13 can be regularly pumped into a transport truck 21 for transfer to a suitable fermentation facility 23. A further possible arrangement is to use transportable storage tanks 13 which can be transported to the ethanol facility 23 when full, emptied at the facility, and returned to the farm.

The embodiment described here is not meant to be restricted to any one type of fermentation process as one skilled in the art will appreciate that a variety fermentation methodologies can be employed for fermenting the collected silage seepage. One skilled in the art will also appreciate that depending on the desired fermentation product, and the profile of the silage seepage, to initiate and/or facilitate the fermentation process, the silage seepage can require the addition of microorganisms and/or hydrolyzing enzymes. In addition, the seepage can be augmented with sugars/carbohydrates or other nutrients to promote, accelerate and/or maintain the process, with the possible added benefit of attaining a higher product (e.g. ethanol) content. Given the exceptionally high BOD of silage seepage, it can be necessary to dilute the seepage prior to fermentation, as dilution may be necessary to reduce the likelihood of inhibitory effects imparted upon the microbial culture. Dilution can be accomplished through the addition of water (e.g. reverse osmosis water collected on site), or may be accomplished by the addition of liquid waste derived from the downstream processing of the fermented seepage. It may also be necessary to provide additional controls (e.g. stirring, gas perfusion, etc. . . .) as will be familiar to one skilled in the art. Furthermore, the fermentation process can be carried out in any number of ways, and can be configured for example for either batch, fed-batch or continuous processing (e.g. continuous culture, chemostat, etc).

As necessary, the desired fermentation process can be facilitated through the addition of naturally existing microorganisms, as well as engineered specialized cultures of prokaryotic and/or eukaryotic origin. For example, yeasts (Saccharomyces cerevisiae) can be used in the production of ethanol, while Clostridium acetobutyricum can be used in the production of butanol. Other microorganisms suitable for production of target products are known, and would be readily apparent to one skilled in the art.

Once the fermentation process is complete the fermented seepage is processed by known methods so as to attain/harvest the desired product. For example, to collect an alcohol product, the fermented seepage can be distilled, after which the alcohol can undergo subsequent processing to attain a target grade for commercial use.

In use, in one particular embodiment representing an example of on-site treatment, the following general procedure is followed. First, the tower silo is loaded with silage tissue and other additives as necessary to promote the desired ensilage process. Guidelines governing moisture content to silage tissue as it pertains to silage seepage is less critical in the present invention, as the seepage represents a collected value by-product. Nevertheless, while increased moisture levels can be readily handled, the target moisture content of the silage tissue is still largely governed by the impacts of the moisture upon the ensilage process, and the desired quality of the resulting ensilage. As the silo begins to produce seepage, the seepage is collected and diverted to a storage tank. As may be necessary, the flow of the seepage is controlled by a valve on the drain line, and as further necessary, the seepage can be filtered. Next, a suitable microbial culture is added to the collected seepage (e.g. yeast for ethanol production). Depending on the targeted fermentation, the storage tank can be subject to further manipulation, such as gas perfusion, mixing, temperature control, etc. As deemed necessary for the targeted fermentation, additional nutrients, enzymes and hydrolyzing solvents can be added to promote the process. The storage tank is then sealed and/or vented as necessary for the particular fermentation process to be carried out. Once the fermentation process is complete, the fermented seepage can be filtered, if necessary and subjected to subsequent downstream processing (e.g. distillation) to collect/harvest the desired product.

Alternatively, the initial fermentation of the silage juice that happens on and within the silage as the silage juice is dripping can produce sufficient levels of alcohol to be of commercial interest to separate or extract without implementing any further fermentation of the silage juice after collection from the silo.

It will be appreciated that, although embodiments have been described and illustrated in detail, various modifications and changes can be made. While several embodiments are described above, some of the features described above can be modified, replaced or even omitted. For example, while the drainage arrangement shown in FIG. 1 is primarily gravity fed, thus not requiring the use of a pump, a pump 19 could be provided along some point in the drain line 11 to facilitate movement of the liquid collected at the bottom of the silo, as shown in FIG. 3B. For example, such a pump could be operated at intervals to move liquid from the drain into a storage tank 13.

It will be appreciated that the silage effluent, whether concentrated or directly collected, is typically rich in nutrients. It can be processed to yield a fertilizer product. As mentioned above, it can be processed to yield alcohol products with or without further fermentation. The effluent can also be aerated to reduce BOD before releasing it to the soil. Aeration of high BOD effluent is known. In particular, concentrated effluent that is obtained by removing water from the raw silage effluent can be expected to have a proportionally higher BOD than the raw effluent, and aeration and/or other treatment to lower the BOD is useful before returning the effluent to the soil either for disposal or as a fertilizer. This is of particular interest when effluent is to be processed at the farm location without trucking it away to a treatment plant.

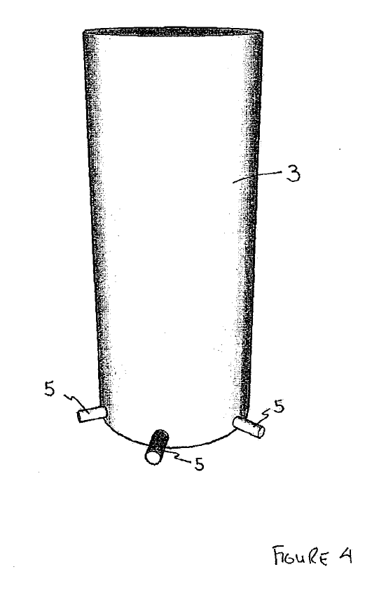

While the above embodiments present a single drain 5 located at the bottom of a conical lower section of the silo, the drain, or a plurality of drains could be placed around the outer circumference of the lower section of the silo, as shown in FIG. 4.

While the storage tank 13 is located at a lower elevation than the silo, and in particular the drain, the storage tank 13 can be located at any suitable location. Where necessary, a suitable pump can be employed to move the silage seepage from the vicinity of the drain to a suitable storage tank 13, particularly in instances where the net movement of drainage from the silo is to a higher elevation compared to the storage tank 13.

While a valve 15 is provided to control the flow of silage seepage from the drain, one skilled in the art will appreciate that such a valve is optional, and that certain installations may not necessitate the use of such a valve.

While a removable filter 17 is provided in the drain line, one skilled in the art will appreciate that certain installations can omit the use of a removable filter, while other scenarios can implement multiple removable filters, depending on the characteristics of the silo seepage being collected.

While the silo in the description provided above is of the tower silo configuration, alternate silo configurations would also be applicable (e.g. horizontal silos).

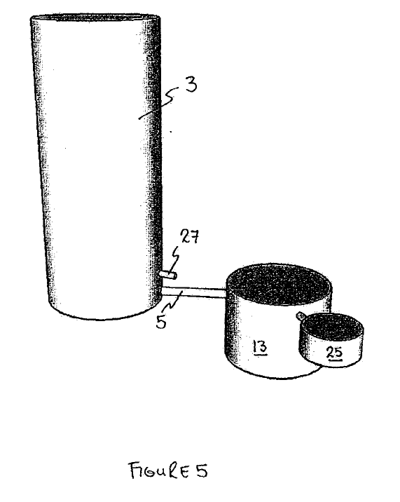

The above arrangement can be altered to include an overflow detector and collector to handle excess seepage that drains from the silo. For example, as shown in FIG. 5, the storage tank 13 could be fitted with a secondary overflow 25 tank to prevent backup and accumulation of silage juice within the silo. The silo itself can be fitted with a secondary drain 27 at a higher elevation than the first primary drain to provide an indication of seepage accumulation within the silo. The arrangement shown excludes the valve and filter assembly for clarity; it will be appreciated that a wide variety of configurations are possible for controlling the flow of seepage from the silo 3. In addition, the secondary drain 27 could also be provided with a valve, to prevent seepage from running through this opening during early seepage collection.

The storage tank 13, the optional secondary overflow tank 25 as well as any other component of the above system can be configured with suitable connectors, access ports, and fittings to allow the components to integrate and operate as described, as well as to provide means to fill, empty, clean, or conduct any other necessary maintenance upon the system. The tanks can also be provided with a measuring device to give the operator an indication of the fill level within the tank. For example, the tanks could be provided with a port that allows for passage of a measuring pole with graduations that define the volume of fluid present in the tank.

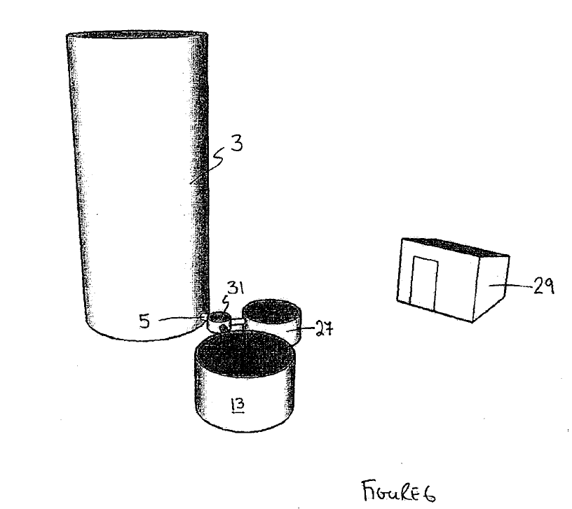

The above arrangement, while operable in a fully manual mode, at least portions of the process can be automated. For example, in instances where a pump is used to move seepage from the drain to the storage tank, the pump can be provided with sensors that detect the accumulation of seepage, and send a signal to a central control unit that instructs the pump to commence pumping. A further example is to provide the storage tank 13 with a sensor that detects when it is full, and subsequently sends a signal to a central control 29 to open a valve 31 to the secondary overflow tank 27. The central control 29 then sends a signal to the valve 31 to divert the flow to the secondary overflow tank 27, and an alarm is activated to indicate a full storage tank. An example of this arrangement is shown in FIG. 6.

Still further alternatives and modifications may occur to those skilled in the art. All such alternatives and modifications are believed to be within the scope of the invention and are covered by the description and Figures appended hereto.

1. A facility for obtaining a fermentation product from silage comprising at least one silage silo having a bottom, a collector at said bottom for collecting a silage juice, and one of a storage tank and separation device in communication with an outlet of said collector.

2. The facility of claim 1, further comprising an overflow tank for collecting overflow seepage from said storage tank.

3. The facility of claim 1, wherein said storage tank is located underground.

4. The facility of claim 1, wherein said facility comprises a reverse osmosis separation device to separate usable water from said silage juice.

5. The facility of claim 1, wherein said drain line comprises a filter assembly for filtering said seepage prior to collection in said storage tank.

6. The facility of claim 1, wherein said storage tank is provided with environmental controls for promoting proper fermentation conditions in said storage tank.

7. The facility of claim 6, where said environmental controls comprise at least one of temperature control, stirring and gas perfusion.

8. The facility of claim 1, wherein said collector comprises a floor pan covering a bottom aperture of a tower silo to prevent egress of seepage from said silo into the surrounding foundation soil.

9. The facility of claim 1, wherein at least one additional pump is provided to move said seepage to said storage tank.

10. The facility of claim 1, further comprising a central processing unit for providing automated control to at least portions of said facility.

11. The facility of claim 1, further comprising a secondary drain at a higher position on said silo for indicating seepage backup and accumulation within said silo.

12. A process for treating silage seepage comprising the steps of collecting said silage seepage generated in a silo, and processing said silage seepage by one of fermentation and separation to produce a value product.

13. The process of claim 12, wherein said value product is alcohol.

14. The process of claim 12, wherein said value produce is ethanol.

15. The process of claim 12, wherein said process comprises the addition of starter cultures.

16. The process of claim 12, wherein said process comprises the addition of hydrolyzing enzymes.

17. The process of claim 12, wherein said process comprises the addition of at least one of sugars, carbohydrates and nutrients to promote said process.

18. The process of claim 12, further comprising the step of diluting said seepage prior to fermentation.

19. The process of claim 18, wherein the step of diluting is accomplished using clean water.

20. The process of claim 18, wherein the step of diluting is accomplished using liquid waste derived from downstream processing of said fermented seepage.

21. The process of claim 12, wherein said processing comprises separating said seepage by reverse osmosis filtration to produce water and concentrated seepage.

22. The process of claim 21, wherein said silage seepage collected from said silo contains about 0.1 to 1% alcohol by weight, and alcohol is separated from said concentrated seepage.

23. The process of claim 21, wherein said water is potable.
